(19) United States
(12) Patent Application Publication
(10) Pub. No.: US 2011/0123632 A1
(43) Pub. Date: May 26, 2011

(54) Nanoscale Adjuvants and Related Pharmaceutical Compositions and Methods

(75) Inventors: Steven S. Kaye, San Diego, CA (US); Whitney Fies, San Diego, CA (US); Yu Liu, San Diego, CA (US)

(73) Assignee: WILDCAT DISCOVERY TECHNOLOGIES, INC., San Diego, CA (US)

(21) Appl. No.: 12/954,410

(22) Filed: Nov. 24, 2010

Related U.S. Application Data
(60) Provisional application No. 61/264,563, filed on Nov. 25, 2009.

(51) Int. Cl. A61K 9/16; A61K 39/00; A61K 9/14; A61K 39/02; A61K 39/12; A61P 35/00; A61P 31/12; A61P 31/04; A61P 37/04

(52) U.S. Cl. 424/491; 424/184.1; 424/489; 424/277.1; 424/234.1; 424/204.1

(57) ABSTRACT

Described herein are nanoscale adjuvants for use in pharmaceutical compositions. In one embodiment, a pharmaceutical composition includes: (a) a vaccine; and (b) a nanoscale adjuvant including an aluminum compound in the form of clusters having a peak size in the sub-micron range.

### CROSS REFERENCE TO RELATED APPLICATION

This application claims the benefit of U.S. Provisional Application No. 61/264,563, filed on Nov. 25, 2009, the disclosure of which is incorporated herein by reference in its entirety.

### FIELD OF THE INVENTION

The invention relates generally to adjuvants. More particularly, the invention relates to nanoscale adjuvants for use in pharmaceutical compositions.

### BACKGROUND

In immunology, a vaccine is a pharmaceutical composition that can improve immune response to a disease. Vaccines can be prophylactic, namely serving to prevent against or reduce the probability of acquiring or developing a disease, or therapeutic, namely serving to treat an existing disease or counteract against further progression of the disease. A vaccine typically includes an antigen, which can stimulate an immune system to more readily recognize and destroy pathogens or other disease-causing agents. In addition to an antigen, a vaccine sometimes includes an adjuvant, which is an agent that can increase an immune response to the antigen, while having little, or no, antigenic effects in itself.

Aluminum compounds are the most widely used adjuvants in vaccines currently in the market. The adjuvanticity of aluminum compounds was first discovered in 1926, and these compounds are recognized as safe by the Food and Drug Administration and international regulatory agencies. While the use of aluminum compounds can increase efficacy of certain vaccines against certain diseases, it would be desirable to increase the adjuvanticity of aluminum compounds so as to provide greater or more extended immunity to those diseases. Also, a number of other vaccines currently do not include aluminum compounds, at least partly because of the limited adjuvanticity of aluminum compounds when used in conjunction with those vaccines.

It is against this background that a need arose to develop the nanoscale adjuvants described herein.

### SUMMARY

Certain embodiments of the invention relate to nanoscale adjuvants having size characteristics that render them desirable for a variety of applications, including immunological applications to increase efficacy or potency of vaccines. In particular, the nanoscale adjuvants can exhibit potent adjuvanticity when used in conjunction with vaccines, thereby providing greater or more extended immunity to diseases. Other embodiments of the invention relate to methods of synthesizing nanoscale adjuvants, such as via ball milling. Further embodiments of the invention relate to methods of using nanoscale adjuvants for immunization against diseases and pharmaceutical compositions including the nanoscale adjuvants.

Other aspects and embodiments of the invention are also contemplated. The foregoing summary and the following detailed description are not meant to restrict the invention to any particular embodiment but are merely meant to describe some embodiments of the invention.

### BRIEF DESCRIPTION OF THE DRAWINGS

For a better understanding of the nature and objects of some embodiments of the invention, reference should be made to the following detailed description taken in conjunction with the accompanying drawings.

FIG. 1 illustrates a cluster size distribution of a conventional aluminum-containing adjuvant in the absence of ball milling, according to an embodiment of the invention.

FIG. 2 illustrates results of measurements of cluster size and cluster size distribution as a function of total milling time, according to an embodiment of the invention.

FIG. 3A and FIG. 3B illustrate results of measurements of cluster size and cluster size distribution as a function of total milling time, according, to an embodiment of the invention.

FIG. 4A and FIG. 4B illustrate results of measurements of cluster size and cluster size distribution when ball milling for a single active period and multiple active periods, according to an embodiment of the invention.

FIG. 5 illustrates the effect of ball milling on crystallite size, according to an embodiment of the invention.

FIG. 6 illustrates results of measurements of cluster size when ball milling at different accelerations, according to an embodiment of the invention.

FIG. 7 illustrates results of measurements or cluster size and cluster size distribution when ball milling with different combinations of materials forming ball bearings and wells, according to an embodiment of the invention.

FIG. 8 illustrates results of measurements of cluster size and cluster size distribution as a function of an adjuvant to ball bearing volume ratio, according to an embodiment of the invention.

FIG. 9 illustrates results of measurements of cluster size when ball milling with different ball hearing sizes, according to an embodiment of the invention.

FIG. 10 illustrates results of measurements of cluster size and cluster size distribution when hall milling under the same ball milling conditions, according to an embodiment of the invention.

FIG. 11 illustrates results of measurements of cluster size and cluster size distribution substantially following ball milling and after storage for about 4 weeks, according to an embodiment of the invention.

FIG. 12A and FIG. 12B illustrate results of an immunological study carried out on differently sized adjuvants using Tetanus toxoid and toxin in mouse models, according to an embodiment of the invention.

FIG. 13 illustrates results of an immunological study carried out on differently sized adjuvants using ovalbumin in mouse models, according to an embodiment of the invention.

### DETAILED DESCRIPTION

### Definitions

The following definitions apply to some of the aspects described with respect to some embodiments of the invention. These definitions may likewise be expanded upon herein.

As used herein, the singular terms “a,” “an,” and “the” include plural referents unless the context clearly dictates otherwise. Thus, for example, reference to an object can include multiple objects unless the context clearly dictates otherwise.

As used herein, the term “set” refers to a collection of one or more objects. Thus, for example, a set of objects can include a single object or multiple objects. Objects of a set also can be referred to as members of the set. Objects of a set can be the same or different. In some instances, objects of a set can share one or more common characteristics.

As used herein, the terms “substantially” and “substantial” refer to a considerable degree or extent. When used in conjunction with an event or circumstance, the ms can refer to instances in which the event or circumstance occurs precisely as well as instances in which the event or circumstance occurs to a close approximation, such as accounting for typical tolerance levels or variability of the embodiments described herein.

As used herein, the term “size” refers to a characteristic dimension of an object. Thus, for example, a size of an object that is spherical can refer to a diameter of the object. In the case of an object that is non-spherical, a size of the non-spherical object can refer to a diameter of a corresponding spherical object, where the corresponding spherical object exhibits or has a particular set of derivable or measurable characteristics that are substantially the same as those of the non-spherical object. Thus, for example, a size of a non-spherical object can refer to a diameter of a corresponding spherical object that exhibits light scattering characteristics that are substantially the same as those of the non-spherical object. Alternatively, or in conjunction, a size of a non-spherical object can refer to an average of various orthogonal dimensions of the object. Thus, for example, a size of an object that is a spheroidal can refer to an average of a major axis and a minor axis of the object. When referring to a set of objects as having a particular size, it is contemplated that the objects can have a distribution of sizes around the particular size. Thus, as used herein, a size of a set of objects can refer to a typical size of a distribution of sizes, such as an average size, a median size, or a peak size.

As used herein, the term “sub-micron range” refers to a general range of dimensions less than about 1 μm or less than about 1,000 nm, such as less than about 999 nm, less than about 900 nm, less than about 800 nm, less than about 700 nm, less than about 600 nm, less than about 500 nm, less than about 400 nm, less than about 300 nm, or less than about 200 nm, and down to about 1 nm or less. In some instances, the term can refer to a particular sub-range within the general range, such as from about 1 nm to about 100 nm, from about 100 nm to about 200 nm, from about 200 nm to about 300 nm, from about 300 nm to about 400 nm, from about 400 nm to about 500 nm, from about 500 nm to about 600 nm, from about 600 nm to about 700 nm, from about 700 nm to about 800 nm, from about 800 nm to about 900 nm, from about 900 nm to about 999 nm, from about 10 nm to about 900 nm, from about 10 nm to about 800 nm, from about 10 nm to about 700 nm, from about 10 nm to about 600 nm, from about 10 nm to about 500 nm, from about 10 nm to about 400 nm, from about 10 nm to about 300 nm, from about 10 nm to about 200 nm, or from about 10 nm to about 100 nm.

### Nanoscale Adjuvants

Certain embodiments of the invention relate to nanoscale adjuvants that are desirable for a variety of applications, including immunological applications to increase efficacy or potency of vaccines. In particular, the nanoscale adjuvants can exhibit potent adjuvanticity when used in conjunction with vaccines, thereby providing greater or more extended immunity to diseases.

Without wishing to be bound by a particular theory, the increased adjuvanticity of nanoscale adjuvants can at least partially derive from their size characteristics. Aluminum compounds currently used as adjuvants typically include micron-sized particles, which correspond to aggregates of crystallites or grains in the form of clusters having sizes on the order of 1 μm. A particle size of an adjuvant can be an important characteristic affecting an immune response to an antigen, and, in the case of certain aluminum compounds, sizes of clusters, rather than sizes of constituent crystallites or grains, can play a greater role in the extent of that immune response. In particular, sizing clusters below about 1 μm and into the sub-micron range can increase their adjuvanticity by mimicking biological activities typically associated with microorganisms, pathogens, or other disease-causing agents of comparable sizes. Without wishing to be hound by a particular theory, clusters having sizes in the sub-micron range can yield potent adjuvanticity based on one or more of the following mechanisms: (1) increased ability to carry antigens (which can be adsorbed onto or otherwise coupled to exposed surfaces of the clusters) into local draining lymph nodes; (2) increased uptake of the antigens by antigen-presenting cells; and (3) increased activation of the antigen-presenting cells.

Size characteristics of nanoscale adjuvants can provide additional benefits. In particular, sizing clusters below about 1 μm and into the sub-micron range can facilitate sterilization of a nanoscale adjuvant by filtration, without requiring heating operations that can increase manufacturing costs and adversely impact the adjuvanticity of the nanoscale adjuvant. Also, the increased ability of a nanoscale adjuvant to carry or promote update of antigens can yield a desired immune response with a reduced amount of the antigens, thereby affording a reduction in costs.

According to some embodiments of the invention, a nanoscale adjuvant includes a set of aluminum compounds that are in the form of clusters having sizes in the sub-micron range. In particular, the clusters can have a distribution of sizes, and a typical size of the distribution of sizes, such as an average size, a median size, or a peak size, can be in the sub-micron range, such as from about 1 nm to about 999 nm. In the case of certain vaccines, the typical cluster size can be fine-tuned or optimized in accordance with a particular trend or relationship relative to adjuvanticity, such as to reduce or minimize the typical cluster size in the case of an inverse relationship between cluster size and adjuvanticity. Thus, for example, the typical cluster size can be less than about 800 nm, less than about 700 nm, less than about 600 nm, less than about 500 nm, less than about 400 nm, less than about 300 nm, or less than about 200 nm, and down to about 10 nm or less. In the case of other vaccines, the typical cluster size can be fine-tuned or optimized in accordance with a particular “sweet-spot” relative to adjuvanticity, such as to adjust the typical cluster size to substantially match an optimal cluster size for adjuvanticity. Thus, for example, the typical cluster size can be in the range of about 1 nm to about 100 nm for one vaccine, in the range of about 100 nm to about 200 nm for another vaccine, in the range of about 200 nm to about 300 nm for another vaccine, in the range of about 300 nm to about 400 nm for another vaccine, in the range of about 400 nm to about 500 nm for another vaccine, in the range of about 500 nm to about 600 nm for another vaccine, in the range of about 600 nm to about 700 nm for another vaccine, in the range of about 700 nm to about 800 nm for another vaccine, in the range of about 800 nm to about 900 nm for another vaccine, and in the range of about 900 nm to about 999 nm for yet another vaccine. It is also contemplated that a distribution of cluster sizes can be multi-modal, in which case the distribution of cluster sizes can have multiple peak sizes. Thus, for example, one peak size can be fine-tuned or optimized in accordance with one particular “sweet-spot” relative to adjuvanticity, another peak size can be fine-tuned or optimized in accordance with another particular “sweet-spot” relative to adjuvanticity, and so forth.

In addition to line-tuning or optimizing a typical cluster size of a distribution of cluster sizes, a spread of the distribution of cluster sizes also can be fine-tuned or optimized, such as in terms of a dispersion or variability relative to the typical cluster size. In the case of certain vaccines, a narrow distribution of cluster sizes can be desirable, such that a large fraction of clusters are suitably sized in accordance with a particular trend or relationship relative to adjuvanticity or in accordance with a particular “sweet-spot” relative to adjuvanticity. Thus, for example, a standard deviation of the distribution of cluster sizes can be less than about 400 nm, less than about 300 nm, less than about 250 nm, less than about 200 nm, or less than about 150 nm, and down to about 50 nm or less. As can be appreciated, a standard deviation also can be represented relative to a typical cluster size, and can be less than about 70%, less than about 60%, less than about 55%, less than about 50%, or less than about 45%, and down to about 10% or less relative to the typical cluster size. It is also contemplated that a distribution of cluster sizes can be multi-modal, in which case each mode of the distribution of cluster sizes can have a spread that is fine-tuned or optimized relative to adjuvanticity.

A nanoscale adjuvant according to some embodiments of the invention can be synthesized via a conversion of a starting material into the nanoscale adjuvant at high yields and at moderate temperatures and pressures. The synthesis can be represented with reference to the formula:

Starting Material→Nanoscale Adjuvant (I)

In formula (I), a starting material can include a set of aluminum compounds. Examples of suitable aluminum compounds include conventional aluminum-containing compounds used as adjuvants in certain vaccines, such as aluminum hydroxide (e.g., Al(OH)₃), aluminum phosphate (e.g., AlPO₄), and alum (e.g., KAl(SO₄)₂.12H₂O). Aluminum hydroxide is typically crystalline, and is typically positively charged at physiological pH, with an isoelectric point (“pI”) of about 11. Aluminum phosphate is typically amorphous, and is typically negatively charged at physiological pH, with a pI between about 5 and about 7. Alum is typically crystalline, and is typically negatively charged at physiological pit, with a pI between about 0.3 and about 0.6. An aluminum compound can be provided as a colloidal suspension, such as in the form of a gel. As noted above, an aluminum compound, in its conventional form as an adjuvant, typically includes micron-sized clusters that are aggregates of crystallites or grains. While certain embodiments are described in terms of using aluminum compounds as starting materials, it is contemplated that other types of starting materials can be subjected to the synthesis according to formula (I) to yield nanoscale adjuvants.

The synthesis according to formula (I) can be carried out using a variety of agitation techniques. One particularly desirable technique is ball milling, in which a starting material undergoes repeated collisions with grinding ball bearings, causing deformation and fracture that can result in structural changes in the starting material. As a result of ball milling, original, micron-sized clusters in the starting material can be at least partially broken apart, thereby yielding modified clusters having sizes in the sub-micron range. Advantageously, ball milling can be carried out to provide a high degree of control over sizes of the resulting clusters, in terms of a typical size of a distribution of those sizes, a dispersion or variability relative to that typical size, or both. This high degree of control can be achieved in a highly reproducible manner and while avoiding or reducing chemical composition changes and crystallite-level or grain-level structural changes, which can adversely impact the adjuvanticity of a resulting nanoscale adjuvant. In particular, ball milling can be carried out, with little, or no, impurities being introduced and with little, or no, changes to a chemical composition, a surface chemistry, and sizes of constituent crystallites or grains. Furthermore, the resulting nanoscale adjuvant is highly stable, such that desirable cluster size characteristics resulting from ball milling are substantially retained over an expected shelf-life or an expected storage period of the nanoscale adjuvant. While certain techniques have been developed to synthesize nanometer-sized particles formed of materials such as gold or polystyrene, those techniques cannot be readily applied to aluminum compounds, and, in particular, cannot be readily applied to synthesize clusters of crystallites or grains having the increased adjuvanticity as described herein.

Ball milling can be carried out using a suitable ball milling device, such as a planetary ball mill, a centrifugal ball mill, an attritor mill, or a shaker mill, which can operate in an inert gas atmosphere, such as one including helium, neon, argon, krypton, xenon, or a combination thereof, or in a reactive gas atmosphere, such as one including a reactive component that contributes to structural changes. The operation of an attritor mill, such as a batch attritor or a horizontal attritor, can involve a mechanical grinding process in which a starting material undergoes repeated collisions with an internally agitated, expanding grinding media. Suitable grinding media include those formed of ceramic, glass, plastic, and steel. The operation of a shaker mill, such as a shaker ball mill, can involve a mechanical milling process in which a starting material undergoes repeated collisions with grinding media while being subjected to repeated vibrations in multiple, substantially orthogonal directions.

Ball milling can be carried out in accordance with a set of conditions, such as: (1) total milling time, which can be in the range of about 0.05 hr to about 12 hr, such as from about 0.1 hr to about 8 hr or from about 0.2 hr to about 6 hr; (2) acceleration, which can be in the range of about 1 g to about 60 g, such as from about 5 g to about 50 g or from about 10 g to about 40 g; (3) materials forming ball bearings and wells, such as ceramics, polymers, metals, metal alloys, lead, antimony, and combinations thereof; (4) adjuvant to ball bearing volume ratio (per well), which can be in the range of about 0.1 to about 100, such as from about 0.2 to about 20 or from about 0.5 to about 15; and (5) ball bearing size, which can be in the range of about 0.1 mm to about 50 mm or more, such as from about 1 mm to about 20 mm or from about 2 mm to about 15 mm. It is contemplated that ball milling conditions can take on other values outside of the particular ranges specified above, such as when scaling ball milling from a batch scale to an industrial scale, or vice versa.

Cluster size characteristics of a resulting nanoscale adjuvant can depend upon a particular set of ball milling conditions used to carry out the synthesis according to formula (I). Accordingly, the cluster size characteristics can be fine-tuned or optimized by proper selection and control over ball milling conditions. In some instances, the selection and control over ball milling conditions can be carried out to vary the cluster size characteristics in accordance with a particular trend or relationship relative to adjuvanticity, such as to reduce or minimize a typical cluster size in the case of an inverse relationship between cluster size and adjuvanticity. In other instances, the selection and control over ball milling conditions can be carried out to vary the cluster size characteristics in accordance with a particular “sweet-spot” relative to adjuvanticity, such as to adjust a typical cluster size to substantially match an optimal cluster size for adjuvanticity.

For example, in the case of certain nanoscale adjuvants when used in conjunction with certain vaccines, a particularly desirable total milling time can be at least about 0.2 hr, such as at least about 0.25 hr or at least about 0.5 hr, and up to about 4 hr, such as up to about 3 hr or up to about 2.5 hr, which can yield a significant reduction in size of clusters while avoiding or reducing heat-induced reaggregation effects that can sometimes result from extended milling time periods. As another example, in the case of certain nanoscale adjuvants when used in conjunction with certain vaccines, a particularly desirable acceleration can be at least about 15 g, such as at least about 20 g or at least about 25 g, and up to about 40 g or more, which can yield a greater reduction and a greater rate of reduction in size of clusters relative to certain other values of the acceleration. As another example, in the case of certain nanoscale adjuvants when used in conjunction with certain vaccines, a particularly desirable adjuvant to ball bearing volume ratio can be up to about 2, such as from about 0.2 to about 2 or from about 0.5 to about 1.7, or can be at least about 5, such as from about 5 to about 15 or from about 5.5 to about 15, which can yield a greater reduction and a greater rate of reduction in size of clusters relative to other certain other values of the number of ball bearings. As a further example, particularly desirable materials forming ball bearings and wells can include ceramics, such as oxides (e.g., alumina or zirconia) carbides, borides, nitrides, silicides, and composites; and polymers, such as acetal or polyoxymethylene (e.g., in homopolymer form or copolymer form), polytetrafluoroethylene, polyetheretherketone, polyphenylene sulfide, polypropylene, ultra-high molecular weight polyethylene, and acrylonitrile butadiene styrene polymers, which can introduce reduced levels of impurities relative to certain other materials. Also, in the case of ball bearings, the use of polymers can yield lighter structures, which can yield a greater reduction and a greater rate of reduction in size of clusters relative to certain other materials.

Depending upon ball milling conditions used, a resulting nanoscale adjuvant can have a chemical composition that substantially corresponds to that of a starting material. In other words, ball milling can be carried out such that a chemical composition of the starting material is substantially retained in the resulting nanoscale adjuvant. For example, the resulting nanoscale adjuvant can include an aluminum compound such as aluminum hydroxide (e.g., Al(OH)₃), aluminum phosphate (e.g., AlPO₄), or alum (e.g., KAl(SO₄)₂.12H₂O), albeit with modified clusters having sizes in the sub-micron range. However, it is contemplated that a chemical composition of a resulting nanoscale adjuvant can vary from that of a starting material. Also, in the case of a combination of different types of aluminum compounds as a starting material, it is contemplated that ball milling can yield an alloy of the aluminum compounds, a blend of the aluminum compounds, or both.

Other techniques can be used to carry out the synthesis according to formula (I), including other agitation techniques such as high-speed overhead stirring and sonication. For example, sonication can be carried out by applying sound energy to a starting material at a frequency of at least about 20 kHz, such as ultrasonic energy in the range of about 20 kHz to about 10 MHz, and at a sonication intensity of at least about 2 W, such as in the range of about 2 W to about 50,000 W, in the range of about 2 W to about 100 W, in the range of about 2 W to about 65 W, or at a higher intensity up to about 50,000 W. Sonication can be carried out for a sonication time of at least about 1 sec, such as in the range of about 1 sec to about 10 min.

### Uses of Nanoscale Adjuvants

The nanoscale adjuvants described herein can be used for a variety of applications, ranging from immunological applications to increase efficacy or potency of vaccines and pharmacological applications to increase efficacy or potency of drugs.

For example, the nanoscale adjuvants can be substituted in place of, or used in conjunction with, conventional adjuvants for pharmaceutical compositions including vaccines. Several types of vaccines can benefit from the nanoscale adjuvants, including live, attenuated vaccines, inactivated vaccines, subunit vaccines, toxoid vaccines, conjugate vaccines. DNA vaccines, and recombinant vector vaccines. Live, attenuated vaccines typically include antigens corresponding to live, but weakened, pathogens; inactivated vaccines typically include antigens corresponding to pathogens that are inactivated by the application of chemicals, heal, or radiation; subunit vaccines typically include antigens corresponding to fragments of pathogens, such as epitopes corresponding to a protein or a protein subunit; toxoid vaccines are typically prepared by inactivating toxic compounds secreted by pathogens to form toxoids; conjugate vaccines are typically prepared by linking a protein, a protein subunit, or a toxoid with a polysaccharide derived from outer coatings of pathogens; DNA vaccines typically operate by insertion and expression of genetic material derived from pathogens; and recombinant vector vaccines also typically operate based on genetic material derived from pathogens, but using a virus or a bacterium as a carrier for the genetic material.

Specific examples of vaccines include those currently used in conjunction with conventional aluminum-based adjuvants, including monovalent vaccines, such as diphtheria vaccines, tetanus vaccines, pertussis vaccines, Haemophilus influenzae type B conjugate vaccines, pneumococcal conjugate vaccines, Hepatitis A vaccines, hepatitis B vaccines, Lyme disease vaccines, anthrax vaccines, rabies vaccines, and veterinary vaccines, as well as multivalent vaccines, such as diphtheria/tetanus vaccines, diphtheria/tetanus/pertussis vaccines, diphtheria/tetanus/pertussis) Haemophilus influenzae type B vaccines, and Hepatitis B/Haemophilus influenzae type B vaccines. Additional examples of vaccines include, those not currently used in conjunction with conventional aluminum-based adjuvants, such as live viral vaccines, inactivated polio vaccines, influenza vaccines, yellow fever vaccines, Japanese encephalitis vaccines, adenovirus vaccines, pneumococcal polysaccharide vaccines, typhoid vaccines, plague vaccines, cholera vaccines, tuberculosis vaccines (or Bacillus Calmette-Guérin vaccines), and meningococcal vaccines.

Also, the nanoscale adjuvants can be used in conjunction with therapeutic vaccines. Therapeutic vaccines include those designed to stimulate an immune response to disease-causing cells or diseased cells, such as cancer cells or cells infected with a disease, or those including antigens derived from such cells, such as tumor antigens derived from cancer cells. Examples of therapeutic vaccines that can benefit from the nanoscale adjuvants include vaccines to treat breast cancer (e.g., by modification of levels of human epidermal growth factor receptor 2 (“HER2”) to treat HER2-positive breast cancer), vaccines to treat advanced non-small cell lung cancer (“NSCLC”) (e.g., epidermal growth factor (“EGF”)-based vaccines), vaccines to treat melanoma (e.g., tyrosinase-related protein 2 (“TRP2”)-based vaccines), vaccines to treat colon cancer, vaccines to treat kidney cancer, vaccines to treat prostate cancer, and vaccines to treat other types of cancers. Additional examples of therapeutic vaccines include those designed to treat immune diseases, such as vaccines to treat acquired immune deficiency syndrome (“AIDS”).

In preparing a pharmaceutical composition, a set of nanoscale adjuvants can be combined, mixed, or otherwise placed in contact with a set of antigens, which can be derived from bacteria, viruses, fungi, cancer cells, or a combination thereof. For example, a nanoscale adjuvant can be added to an antigen to form an adjuvanted vaccine, which can be isolated and subjected to other suitable treatment for inclusion in a pharmaceutical composition. As another example, an antigen can be added to a nanoscale adjuvant, which can be in the form of a colloidal suspension. Different types of nanoscale adjuvants can be included, such as one nanoscale adjuvant including a particular aluminum compound and having a particular size or size distribution, another nanoscale adjuvant including another particular aluminum compound and having another particular size or size distribution, and so forth. In the case of multivalent vaccines, each type of nanoscale adjuvant can have a particular size or size distribution that is optimized or otherwise tailored for a respective antigen to increase an immune response to that antigen. Different types of nanoscale adjuvants can be combined individually in series in any suitable order, or can be combined at once or in groups. Similarly and in the case of multivalent vaccines, different types of antigens can be combined individually in series in any suitable order, or can be combined at once or in groups. In some instances, antigens and their respective nanoscale adjuvants can be combined in pairs, and the antigen/adjuvant pairs can be combined at once or in groups.

Once combined, an antigen and a nanoscale adjuvant can be coupled based on an attractive interaction. Coupling between an antigen and a nanoscale adjuvant can be based on, for example, adsorption, covalent bonding, hydrogen bonding, ionic bonding, van der Waals bonding, or a combination thereof. For example, an antigen can be adsorbed on exposed surfaces of clusters included in a nanoscale adjuvant. Because of porosity or texturing created by constituent grains or crystallites, the clusters can have a large surface area to weight ratio and a high capacity to adsorb an antigen, such that, for example, at least about 50 wt. %, at least about 60 wt. %, at least about 70 wt. %, at least about 80 wt. %, or at least about 90 wt. %, and up to about 95 wt. %, up to about 99 wt. %, or up to about 99.5 wt. % or more of the antigen can be adsorbed. Clusters included in a nanoscale adjuvant also can be surface functionalized to increase or modify an attractive interaction with an antigen.

As included in a resulting pharmaceutical composition, an amount of a set of nanoscale adjuvants can be represented in terms of a weight of aluminum (or other elemental component) per dose of the composition, which can be in the range of about 1 μg/dose to about 1.5 mg/dose, such as from about 10 μg/dose to about 1.25 mg/dose, from about 50 μg/dose to about 850 μg/dose, from about 100 μg/dose to about 850 μg/dose, from about 200 μg/dose to about 850 μg/dose, from about 300 μg/dose, to about 850 μg/dose, from about 400 μg/dose to about 850 μg/dose, or from about 500 μg/dose to about 850 μg/dose. A dose of a pharmaceutical composition can include an amount of a set of antigens in the range of about 1 μg to about 25 μg, and can have a volume in the range of about 0.1 ml to about 1 ml, such as from about 0.1 ml to about 0.2 ml, about 0.25 ml, about 0.5 ml, or about 1 ml, although other immunologically effective doses are also contemplated. It is also contemplated that dosage amounts can be suitably adjusted within or outside of the particular ranges specified above, depending upon whether the pharmaceutical composition is intended for a human patient or a non-human patient, and, in the case of a human patient, whether the patient is an adult or a child.

A pharmaceutical composition can include additional components, such as a set of excipients and a set of buffers. Examples of buffers include a phosphate buffer, a Tris buffer, a borate buffer, a succinate buffer, a histidine buffer, a citrate buffer, and combinations thereof. The inclusion of buffers can maintain a desirable pH for the composition, which can be in the range of about 5 to about 8, such as from about 5.5 to about 7.5 or from about 6 to about 7.

Once prepared, a pharmaceutical composition can be administered to a patient, so as to improve immunity of the patient against developing or acquiring a particular disease or to ameliorate effects or symptoms of that disease. The patient can be a human patient or a non-human patient, and the composition can be administered in a variety of ways, such as orally, via inhalation, intravenously, intramuscular injection, or a combination thereof.

Advantageously, the use of nanoscale adjuvants can increase efficacy or potency of vaccines, relative to the absence of the nanoscale adjuvants or relative to the use of conventional adjuvants. This increase in efficacy can be determined based on immunological studies, such as increased survival probabilities as observed in non-human models, reduced probabilities of developing diseases as observed in non-human models or in human patients, increased levels of antibodies or faster rate of production of antibodies by an immune system, or a combination thereof.

### EXAMPLES

The following examples describe specific aspects of some embodiments of the invention to illustrate and provide a description for those of ordinary skill in the art. The examples should not be construed as limiting the invention, as the examples merely provide specific methodology useful in understanding and practicing some embodiments of the invention.

### Example 1

### Methodology for Synthesis of Nanoscale Adjuvants and Characterization of Starting Materials and Nanoscale Adjuvants

Nanoscale adjuvants were synthesized from starting materials corresponding to commercially available aluminum hydroxide hydrogels (2 wt. % Alhydrogel, Cedarlane Laboratories Ltd.). The starting materials were subjected to ball milling using a device corresponding to a PM 400 planetary ball mill (Retsch® GmbH). Ball milling was carried out under a variety of ball milling conditions to determine the effect (if any) of those conditions on characteristics of resulting nanoscale adjuvants. For example, hall milling was carried out for a total milling time between about 0 hr and about 5 hr, at an acceleration between about 13 g and about 38 g, with a number of ball bearings between about 10 and about 60, with a ball bearing size between about 2 mm and about 8 mm, and with different combinations of materials forming ball bearings and wells. In some instances, the total milling time included multiple active milling periods, which were interrupted by pause or rest periods, such as with active milling periods between about 5 min and about 15 min and with comparable rest periods.

Size characteristics of starting materials and resulting nanoscale adjuvants were measured using a laser particle size analysis device (Saturn DigiSizer® 5200, Micromeritics Instrument Corp.). In particular, samples were dispersed in aqueous media and circulated through the path of a laser beam. As the samples passed through the laser beam, light scattering occurred at angles and intensities related to a particle size. Analysis of scattered light was based on the Mie theory for scattering by spherical particles, and reported size data was based on corresponding spherical particles exhibiting comparable light scattering characteristics.

### Example 2

### Characterization of Starting Material

A commercially available aluminum hydroxide hydrogel typically includes crystalline aluminum hydroxide in the form of smaller particles that aggregate to forth larger particles. In particular, the smaller particles typically correspond to crystallites or grains, and the larger particles typically correspond to clusters of crystallites and having sizes on the order of 1 μm. As further described herein, ball milling can be carried out to at least partially break apart the clusters in a controlled fashion to yield a reduction in size of the clusters.

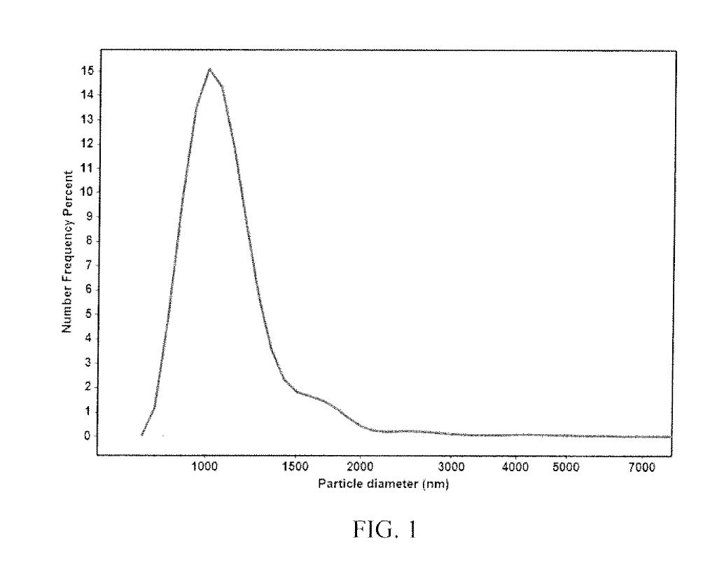

To determine size characteristics of clusters in the absence of or prior to, ball milling, a commercially available aluminum hydroxide hydrogel was subjected to measurements in accordance with the methodology set forth in Example 1. Results of the measurements are illustrated in FIG. 1 and are based on circulating a sample of about 7 ml of the commercially available aluminum hydroxide hydrogel. As can be appreciated with reference to FIG. 1, the sample included clusters with a typical size (e.g., a peak size or a maximum number frequency diameter) of about 1,005 nm and with a majority of the clusters sized between about 1 μm and about 2 μn.

### Example 3

### Synthesis and Characterization of Nanoscale Adjuvants—Effect of Total Milling Time

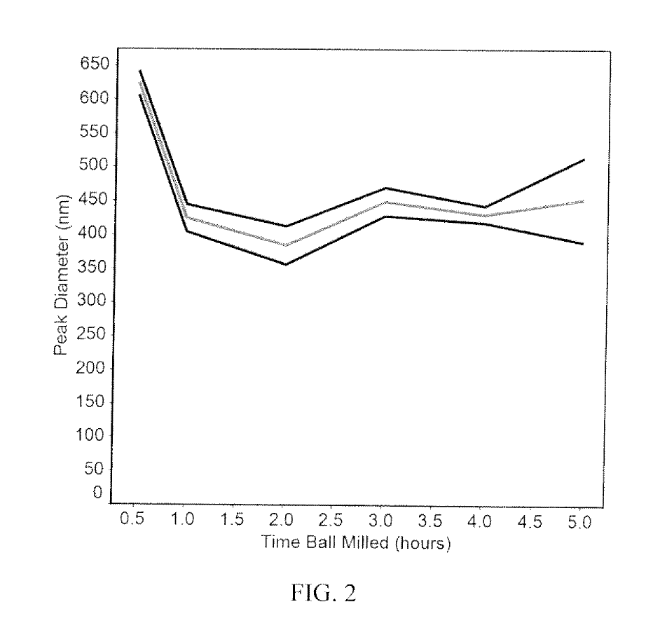

Nanoscale adjuvants were synthesized via ball milling and characterized in accordance with the methodology set forth in Example 1. To determine the effect of total milling time on cluster size and cluster size distribution, samples each including about 7 ml of a commercially available aluminum hydroxide hydrogel were subjected to ball milling for different total milling times, while other ball milling conditions were held substantially constant across the samples. Following ball milling, size characteristics of resulting nanoscale adjuvants were measured. Results of the measurements are illustrated in FIG. 2 and are based on ball milling at an acceleration of about 23 g. In particular, three line graphs are illustrated in FIG. 2, with a middle line graph representing a typical size (e.g., a peak size or a maximum number frequency diameter) versus total milling time, and with an upper line graph and a lower line graph representing a dispersion or variability about the typical size (e.g., a vertical distance between the upper line graph and the lower line graph corresponding to a standard deviation of a distribution of sizes).

As can be appreciated with reference to FIG. 2, a sample milled for about 0.5 hr exhibited a significant reduction in size of clusters, with a typical size of about 625 nm relative to about 1,005 nm prior to ball milling or a reduction of about 40% within about 0.5 hr. Further ball milling beyond about 0.5 hr and up to about 2 hr yielded additional reductions in size of clusters, albeit with more moderate reductions per unit time interval and with a tapering of those reductions as a typical size approached towards a “steady state” or “equilibrium” value. In particular, a sample milled for about 1 hr had a typical size of about 425 nm, and a sample milled for about 2 hr had a typical size of about 383 nm. Without wishing to be bound by a particular theory, this observed trend indicates that ball milling has effectively broken apart original clusters sized on the order of 1 μm and has shifted a cluster size distribution towards a value of about 400 nm within a milling time period of about 2 hr. Samples that were milled beyond about 2 hr exhibited a slight increase in size of clusters, and, for example, a sample milled for about 5 hr had a typical size of about 425 nm. Without wishing to be bound by a particular theory, this observed increase in cluster size may result from heat-induced reaggregation when ball milling for extended milling time periods.

In addition to controllably reducing a typical size of clusters, ball milling also afforded control over a dispersion or variability about the typical size. As can be appreciated with reference to FIG. 2, ball milling for up to about 4 hr yielded a narrow distribution of cluster sizes, with a majority of clusters sized within a range of about 100 nm (or less) relative to a typical size. Further ball milling beyond about 4 hr yielded a slight increase in dispersion of cluster sizes (in terms of absolute value). Without wishing to be hound by a particular theory, this observed increase in cluster size dispersion may result from heat-induced reaggregation when ball milling for extended milling time periods.

### Example 4

### Synthesis and Characterization of Nanoscale Adjuvants—Effect of Total Milling Time

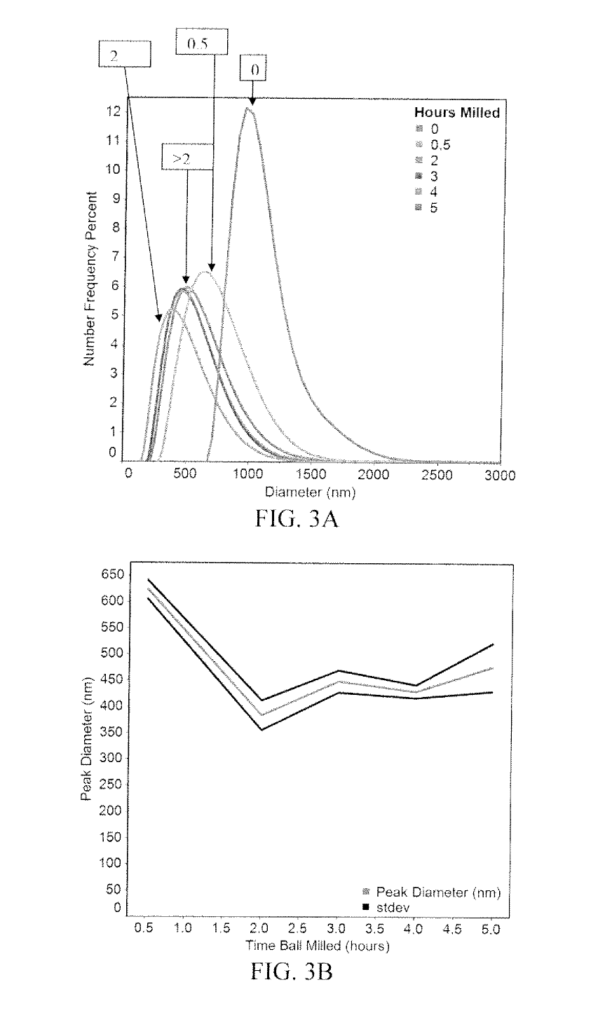

Nanoscale adjuvants were synthesized via ball milling and characterized in accordance with the methodology set forth in Example 1. To determine the effect of total milling time on cluster size and cluster size distribution, samples each including about 7 ml of a commercially available aluminum hydroxide hydrogel were subjected to ball milling for different total milling times, while other ball milling conditions were held substantially constant across the samples. Following ball milling, size characteristics of resulting nanoscale adjuvants were measured. Results of the measurements are illustrated in FIG. 3A and FIG. 3B and are based on ball milling at an acceleration of about 37 g, with 10 ball bearings formed of polyoxymethylene (Delrin®, Dupont™), and with wells formed of zirconia. Six cluster size distributions are illustrated in FIG. 3A, with a rightmost distribution corresponding to a starting material that was not subjected to halt milling, and with remaining distributions corresponding to samples that were subjected to ball milling for different total milling times. Three line graphs are illustrated in FIG. 3B, with a middle line graph representing a typical size versus total milling time, and with an upper line graph and a lower line graph representing a dispersion or variability about the typical size.

As can be appreciated with reference to FIG. 3A and FIG. 3B, a sample milled for about 0.5 hr exhibited a significant reduction in size of clusters, with a typical size of about 625 nm relative to about 1,005 nm prior to ball milling or a reduction of about 40% within about 0.5 hr. Further ball milling beyond about 0.5 hr and up to about 2 hr yielded additional reductions in size of clusters, albeit with more moderate reductions per unit time interval and with a tapering of those reductions as a typical size approached towards a “steady state” or “equilibrium” value. In particular, a sample milled for about 1 hr had a typical size of about 540 nm, and a sample milled for about 2 hr had a typical size of about 380 nm. Without wishing to be hound by a particular theory, this observed trend indicates that ball milling has effectively broken apart original clusters sized on the order of 1 μm and has shifted a cluster size distribution towards a value of about 400 nm within a milling time period of about 2 hr. Samples that were milled beyond about 2 hr exhibited a slight increase in size of clusters, and, for example, a sample milled for about 5 hr had a typical size of about 475 nm. Without wishing to be bound by a particular theory, this observed increase in cluster size may result from heat-induced reaggregation when ball milling for extended milling time periods.

As also can be appreciated with reference to FIG. 3A and FIG. 3B, ball milling for up to about 4 hr yielded a narrow distribution of cluster sizes, with a majority of clusters sized within a range of about 100 nm (or less) relative to a typical size. Further ball milling beyond about 4 hr yielded a slight increase in dispersion of cluster sizes (in terms of absolute value). Without wishing to be bound by a particular theory, this observed increase in cluster size dispersion may result from heat-induced reaggregation when ball milling for extended milling time periods.

### Example 5

### Synthesis and Characterization of Nanoscale Adjuvants—Effect of Milling Time

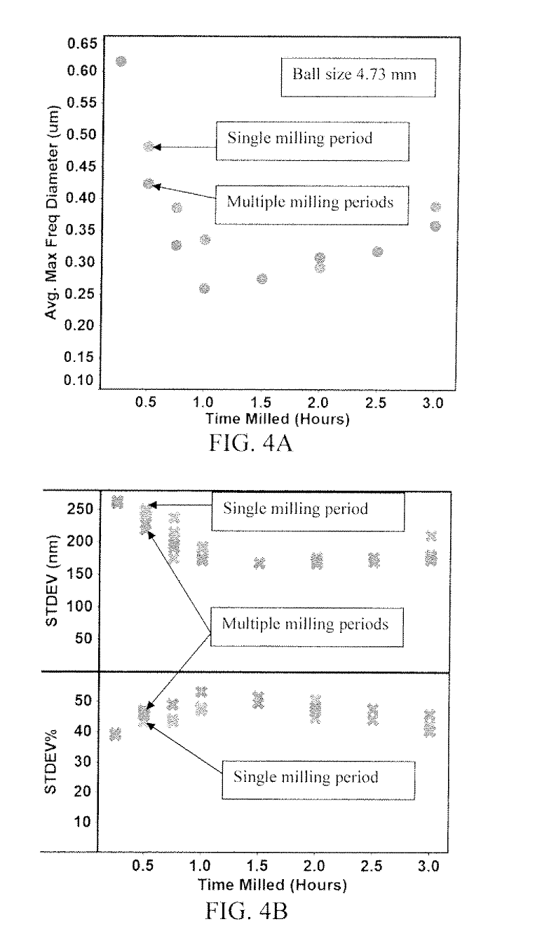

Nanoscale adjuvants were synthesized via ball milling and characterized in accordance with the methodology set forth in Example 1. To determine the effect of milling on cluster size and cluster size distribution, samples each including about 7 ml of a commercially available aluminum hydroxide hydrogel were subjected to ball milling for different total milling times, while other ball milling conditions were held substantially constant across the samples. For each total milling time, a set of samples were subjected to ball milling for a single, substantially continuous, active milling period, while another set of samples were subjected to ball milling for multiple active milling periods of about 15 min each. Following ball milling, size characteristics of resulting nanoscale adjuvants were measured. Results of the measurements are illustrated in FIG. 4A and FIG. 4B and are based on ball milling with a ball bearing size of about 4.73 mm.

As can be appreciated with reference to FIG. 4A and FIG. 4B, ball milling for a single active milling period did not yield significantly different results relative to ball milling for multiple active milling periods. Referring to FIG. 4A, a set of samples milled for about 0.25 hr exhibited a significant reduction in size of clusters, with a typical size of about 625 nm relative to about 1,005 nm prior to ball milling or a reduction of about 40% within about 0.25 hr, and a set of samples milled for about 0.5 hr exhibited another significant reduction in size of clusters, with a typical size between about 425 nm and about 475 nm relative to about 1,005 nm prior to ball milling or a reduction of about 50% within about 0.5 hr. Further hall milling beyond about 0.5 hr and up to about 1 hr yielded additional reductions in size of clusters, albeit with more moderate reductions per unit time interval and with a tapering of those reductions as a typical size approached towards a “steady state” or “equilibrium” value. In particular, a set of samples milled for about 1 hr had a typical size between about 250 nm and about 340 nm. Without wishing to be bound by a particular theory, this observed trend indicates that ball milling has effectively broken apart original clusters sized on the order of 1 μm and has shifted a cluster size distribution towards a value of about 300 nm within a milling time period of about 1 hr. Samples that were milled beyond about 1 hr exhibited a slight increase in size of clusters, and, for example, a sample milled for about 3 hr had a typical size between about 350 nm and about 375 nm. Without wishing to be bound by a particular theory, this observed increase in cluster size may result from heat-induced reaggregation when ball milling for extended milling time periods.

Referring to FIG. 4B, ball milling yielded a narrow distribution of cluster sizes, with a majority of clusters sized within a narrow range relative to a typical size. In particular, the set of samples milled for about 0.25 hr exhibited a standard deviation of about 250 nm (in terms of absolute value) or about 40% (relative to a typical size), and the set of samples milled for about 0.5 hr exhibited a standard deviation between about 210 nm and about 250 nm (in terms of absolute value) or between about 40% and about 50% (relative to a typical size). Further ball milling beyond about 0.5 hr and up to about 3 hr yielded moderate changes in the standard deviation per unit time interval and with a tapering of those changes towards a “steady state” or “equilibrium” value of about 160 nm (in terms of absolute value) or about 45% (relative to a typical size). Samples that were milled beyond about 2.5 hr exhibited a slight increase in the standard deviation (in terms of absolute value), which may result from heat-induced reaggregation.

### Example 6

### Synthesis and Characterization of Nanoscale Adjuvants—Crystallite Size

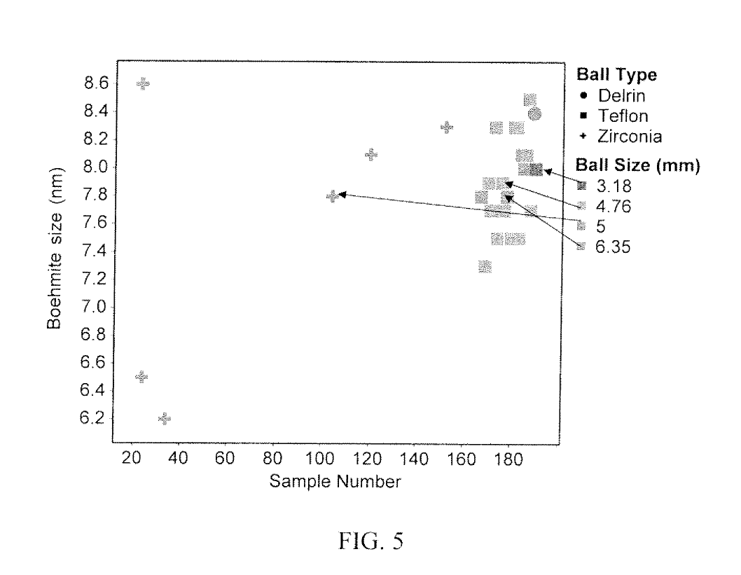

Nanoscale adjuvants were synthesized via ball milling and characterized in accordance with the methodology set forth in Example 1. To determine the effect of ball milling on crystallite size, samples each including about 7 ml of a commercially available aluminum hydroxide hydrogel were subjected to ball milling at different accelerations, with different materials forming ball bearings, and with different ball bearing sizes, while other ball milling conditions were held substantially constant across the samples. Following ball milling, sizes of crystallites within resulting nanoscale adjuvants were measured, and results of the measurements are illustrated in FIG. 5.

As can be appreciated with reference to FIG. 5, ball milling under a variety of ball milling conditions did not yield significant changes in a typical crystallite size relative to that of a starting material, and also did not yield significant changes across samples. In particular, a typical crystallite size remained within a range of about 1.4 nm and between about 7.2 nm to about 8.6 nm across the samples. Ball milling under certain ball milling conditions, such as at an acceleration of about 32 g (or more), did yield a reduction in a typical crystallite size to a range of about 6.2 nm to about 6.4 nm. Without wishing to be bound by a particular theory, this observed behavior indicates that, while ball milling can effectively break apart clusters in a controlled fashion, size characteristics of constituent crystallites remain substantially intact under a variety of hall milling conditions.

### Example 7

### Synthesis and Characterization of Nanoscale Adjuvants—Effect of Acceleration

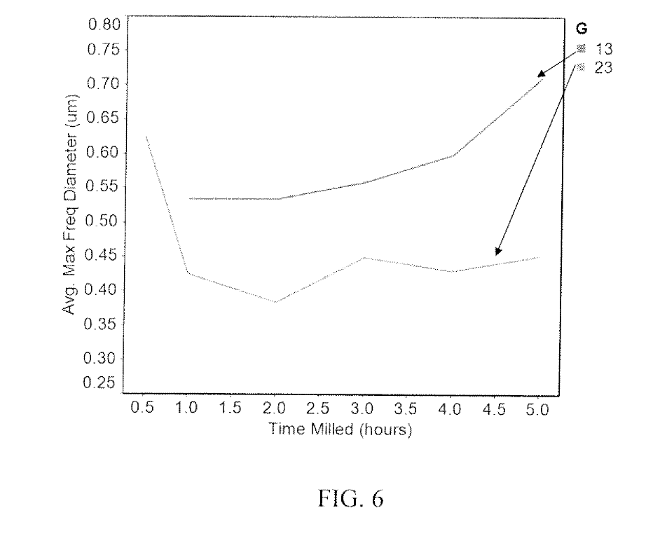

Nanoscale adjuvants were synthesized via ball milling and characterized in accordance with the methodology set forth in Example 1. To determine the effect of acceleration on cluster size, samples each including about 7 ml of a commercially available aluminum hydroxide hydrogel were subjected to ball milling at different accelerations and for different total milling times, while other ball milling conditions were held substantially constant across the samples. Following ball milling, size characteristics of resulting nanoscale adjuvants were measured. Results of the measurements are illustrated in FIG. 6 and are based on ball milling at an acceleration of about 13 g for one set of samples and an acceleration of about 23 g for another set of samples.

As can be appreciated with reference to FIG. 6, a greater acceleration during ball milling yielded a greater reduction and a greater rate of reduction in size of clusters. In particular, a sample milled at about 23 g for about 0.5 hr exhibited a significant reduction in size of clusters, with a typical size of about 625 nm relative to about 1,005 nm prior to ball milling or a reduction of about 40% within about 0.5 hr, and a sample milled at about 23 g for about 1 hr exhibited another significant reduction in size of clusters, with a typical size of about 425 nm relative to about 1,005 nm prior to ball milling or a reduction of about 60% within about 1 hr. Further hall milling at about 23 g beyond about 1 hr yielded moderate changes in size of clusters and eventually a slight increase in size of clusters. By way of contrast, a sample milled at about 13 g for about 1 hr exhibited a smaller reduction in size of clusters, with a typical size of about 530 nm relative to about 1,005 nm prior to ball milling or a reduction of about 50% within about 1 hr. Further ball milling at about 13 g beyond about 1 hr yielded moderate changes in size of clusters and eventually an increase in size of clusters.

### Example 8

### Synthesis and Characterization of Nanoscale Adjuvants—Effect of Materials Forming Ball Bearings and Wells

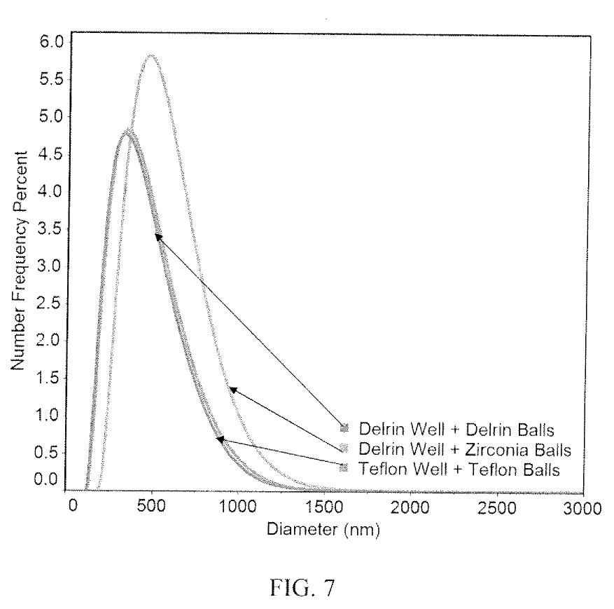

Nanoscale adjuvants were synthesized via ball milling and characterized in accordance with the methodology set forth in Example 1. To determine material effects on cluster size and cluster size distribution, samples each including about 7 ml of a commercially available aluminum hydroxide hydrogel were subjected to ball milling with different combinations of materials forming ball bearings and wells, while other ball milling conditions were held substantially constant across the samples. Following ball milling, size characteristics of resulting nanoscale adjuvants were measured. Results of the measurements are illustrated in FIG. 7 and are based on hall milling at an acceleration of about 23 g, for a total milling time of about 4 hr, and with 10 ball bearings. Three cluster size distributions are illustrated in FIG. 7, with one distribution corresponding to ball hearings and wells formed of polyoxymethylene (Delrin®, Dupont™), another distribution corresponding to ball bearings formed of zirconia and wells formed of polyoxymethylene (Delrin®, Dupont™), and another distribution corresponding to ball hearings and wells formed of polytetrafluoroethylene (Teflon®, Dupont™). In this example, dimensions of ball bearings and wells were held substantially constant, and polymeric structures formed of polyoxymethylene or polytetrafluoroethylene were lighter than corresponding ceramic structures formed of zirconia.

As can be appreciated with reference to FIG. 7, the use of lighter bull bearings formed of polyoxymethylene or polytetrafluoroethylene yielded a significant reduction in size of clusters and a narrow distribution of cluster sizes, in particular, samples milled using polymeric ball bearings had a typical size of about 375 nm relative to about 1,005 nm prior to ball milling or a reduction of about 60%.

### Example 9

### Synthesis and Characterization of Nanoscale Adjuvants—Effect of Materials Forming Ball Bearings and Wells

Nanoscale adjuvants were synthesized via ball milling in accordance with the methodology set forth in Example 1. To determine the level of impurities (if any) introduced by ball milling, samples each including about 7 ml of a commercially available aluminum hydroxide hydrogel were subjected to ball milling with different combinations of materials forming ball bearings and wells. Following ball milling, the level of impurities in resulting nanoscale adjuvants was determined in accordance with conventional techniques.

The use of ball bearings and wells formed of a metal or a metal alloy was sometimes observed to introduce undesirable levels of impurities, which were visually detectable via magnified images. In contrast, the use of corresponding structures formed of a ceramic or a polymer yielded a significant reduction in levels of impurities. Table 1 below sets forth a variety of ball milling conditions and the levels of impurities in resulting samples, expressed in terms of wt. % of Total Organic Carbon (“TOC”), wt. % of other forms of carbon, and a weight ratio of zirconium relative to a total sample weight (if applicable).

TABLE 1

| | | Num- | | | | | |
|---|---|---|---|---|---|---|---|
| | | ber | Time | | | | |
| Well | Ball | of | Milled | G | W/W % | W/W % | μg/g |
| Material | Material | Balls | (hours) | Milled | C (TOC) | C (other) | Zr |
| Delrin | ZrO2 | 10 | 0.5 | 13 | 0.006 | — | 1 |
| Delrin | ZrO2 | 10 | 0.5 | 23 | 0.003 | — | 2 |
| Delrin | ZrO2 | 10 | 0.5 | 38 | 0.005 | — | 5 |
| Delrin | ZrO2 | 10 | 3 | 13 | 0.01 | — | 6 |
| Delrin | ZrO2 | 10 | 3 | 23 | 0.007 | — | 23 |
| Delrin | ZrO2 | 10 | 3 | 38 | 0.005 | — | 18 |
| Delrin | ZrO2 | 30 | 1 | 38 | 0.004 | — | 11 |
| Delrin | ZrO2 | 50 | 1 | 38 | 0.005 | — | 3 |
| Delrin | Delrin | 10 | 0.5 | 38 | 0.002 | — | — |
| Delrin | Delrin | 10 | 3 | 38 | 0.005 | — | — |
| Delrin | Delrin | 10 | 0.5 | 23 | <0.002 | — | — |
| Delrin | Delrin | 10 | 3 | 23 | 0.002 | — | — |
| Delrin | Delrin | 10 | 0.5 | 13 | <0.002 | — | — |
| Delrin | Delrin | 10 | 3 | 13 | <0.002 | — | — |
| Delrin | Delrin | 10 | 5 | 23 | <0.002 | — | — |
| Delrin | Delrin | 10 | 5 in 15 | 23 | <0.002 | — | — |
| | | | min | | | | |
| | | | incre- | | | | |
| | | | ments | | | | |
| Teflon | Teflon | 10 | 0.5 | 38 | <0.002 | <0.005 | — |
| Teflon | Teflon | 10 | 3 | 38 | <0.002 | 0.005 | — |
| Teflon | Teflon | 10 | 0.5 | 23 | <0.002 | <0.005 | — |
| Teflon | Teflon | 10 | 3 | 23 | 0.002 | 0.006 | — |
| Teflon | Teflon | 10 | 0.5 | 13 | <0.002 | <0.005 | — |
| Teflon | Teflon | 10 | 3 | 13 | <0.002 | <0.005 | — |
| Teflon | Teflon | 10 | 5 | 23 | <0.002 | <0.005 | — |
| Teflon | Teflon | 10 | 5 in 15 | 23 | <0.002 | 0.005 | — |
| | | | min | | | | |
| | | | incre- | | | | |
| | | | ments | | | | |

As can be appreciated with reference to Table 1, the use of ball bearings and wells formed of polyoxymethylene (Delrin®, Dupont™) or polytetrafluoroethylene (Teflon®, Dupont™) yielded reduced levels of impurities relative to the use of structures formed of zirconia. In particular, samples milled using polymeric ball bearings and polymeric wells exhibited a wt. % of TOC that was no greater than about 0.002 and a wt. % of other forms of carbon that was no greater than about 0.006.

### Example 10

### Synthesis and Characterization of Nanoscale Adjuvants—Effect of Number of Ball Bearings

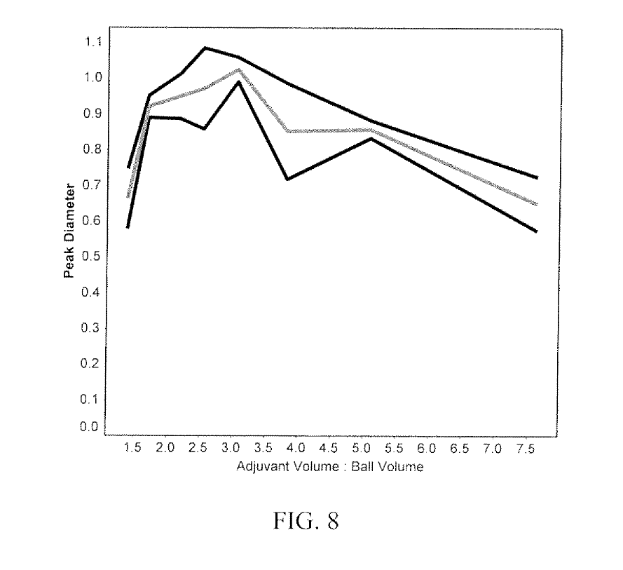

Nanoscale adjuvants were synthesized via ball milling and characterized in accordance with the methodology set forth in Example 1. To determine the effect of a number of ball bearings on cluster size and cluster size distribution, samples each including about 7 ml of a commercially available aluminum hydroxide hydrogel were subjected to ball milling with different numbers of ball bearings, while other ball milling conditions were held substantially constant across the samples. Following ball milling, size characteristics of resulting nanoscale adjuvants were measured. Results of the measurements are illustrated in FIG. 8 and are based on bail milling at an acceleration of about 37 g, for a total milling time of about 1 hr, with ball bearings formed of zirconia, with wells formed of polyoxymethylene (Delrin®, Dupont™), and an adjuvant to ball bearing volume ratio ranging from 1.5 to 7.5. Three line graphs are illustrated in FIG. 8, with a middle line graph representing a typical size versus the adjuvant to ball bearing volume ratio, and with an upper line graph and a lower line graph representing a dispersion or variability about the typical size.

As can be appreciated with reference to FIG. 8, the use of a smaller volume ratio (e.g., 2.0 or less) or a larger volume ratio (e.g., 5.5 or more) yielded a greater reduction in size of clusters, relative to the use of a moderate volume ratio (e.g., between 2.5 and 5.0). In particular, samples milled using a volume ratio of 2.0 (or less) had a typical cluster size of about 850 nm (or less) after milling for about 1 hr, relative to about 1,005 nm prior to hall milling or a reduction of about 15% within about 1 hr, and samples milled using a volume ratio of 5.5 (or greater) had a typical cluster size of about 800 nm (or less) after milling for about 1 hr, relative to about 1,005 nm prior to ball milling or a reduction of about 20% within about 1 hr. By way of contrast, a sample milled using a volume ratio of about 2.2 exhibited a smaller reduction in size of clusters, with a typical size of about 950 nm relative to about 1,005 nm prior to ball milling or a reduction of about 5% within about 1 hr. As also can be appreciated with reference to FIG. 8, samples milled using a larger adjuvant to ball hearing volume ratio typically exhibited a narrower distribution of cluster sizes, relative to the use of a moderate adjuvant to ball bearing volume ratio or a smaller adjuvant to ball bearing volume ratio.

### Example 11

### Synthesis and Characterization of Nanoscale Adjuvants—Effect of Ball Bearing Size

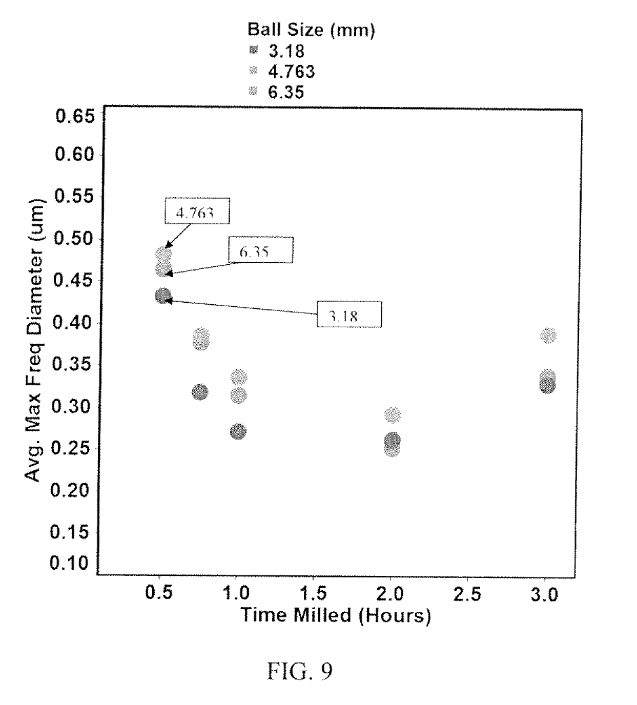

Nanoscale adjuvants were synthesized via ball milling and characterized in accordance with the methodology set forth in Example 1. To determine the effect of ball hearing size on cluster size, samples each including about 7 ml of a commercially available aluminum hydroxide hydrogel were subjected to ball milling with different ball bearing sizes, while other ball milling conditions were held substantially constant across the samples. Following ball milling, size characteristics of resulting nanoscale adjuvants were measured. Results of the measurements are illustrated in FIG. 9 and are based on ball milling with ball bearing sizes of about 3.18 mm, about 4.763 mm, and about 6.35 mm.

As can be appreciated with reference to FIG. 9, hall milling with different ball hearing sizes did not yield significant differences in terms of cluster sizes. Referring to FIG. 9, a set of samples milled for about 0.5 hr had a typical size that varied between about 425 nm and about 480 nm depending on the ball bearing size used, and a set of samples milled for about 1 hr had a typical size that varied between about 275 nm and about 340 nm depending on the ball hearing size used. Further ball milling beyond about 1 hr and up to about 2 hr yielded additional reductions in size of clusters for each ball hearing size used, albeit with more moderate reductions per unit time interval and with a tapering of those reductions as a typical size approached towards a “steady state” or “equilibrium” value. Samples that were ball milled beyond about 2 hr exhibited a slight increase in size of clusters, and, for example, a set of samples milled for about 3 hr had a typical size that varied between about 325 nm and about 390 nm depending on the ball hearing size used.

### Example 12

### Synthesis and Characterization of Nanoscale Adjuvants—Reproducibility

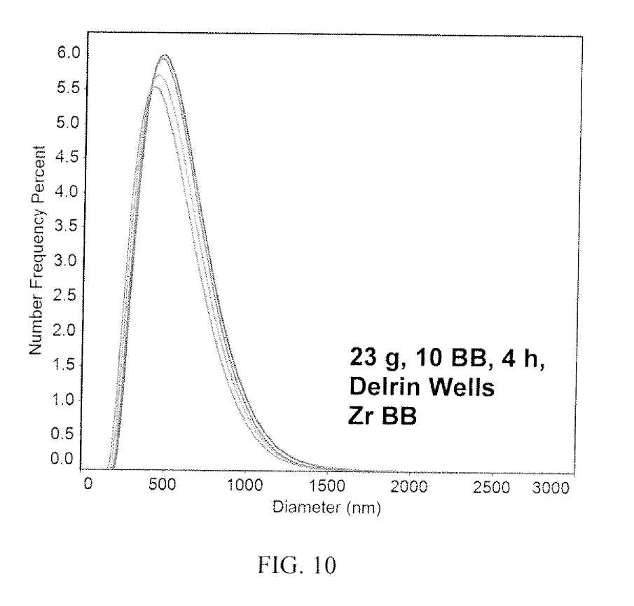

Nanoscale adjuvants were synthesized via hall milling and characterized in accordance with the methodology set forth in Example 1. To determine the reproducibility in terms of cluster size and cluster size distribution, four samples each including about 7 ml of a commercially available aluminum hydroxide hydrogel were subjected to ball milling under the same hall milling conditions. Following ball milling, size characteristics of resulting nanoscale adjuvants were measured. Results of the measurements are illustrated in FIG. 10 and are based on ball milling at an acceleration of about 23 g, for a total milling time of about 4 hr, with 10 ball bearings formed of zirconia, and with wells formed of polyoxymethylene (Delrin®, Dupont™). Four cluster size distributions are illustrated in FIG. 10, with each distribution corresponding to a respective one of the four samples.

As can be appreciated with reference to FIG. 10, ball milling under the same ball milling conditions yielded highly reproducible results in terms of cluster size and cluster size distribution. Referring to FIG. 10, the four samples had typical sizes that were closely similar and that varied within a range of less than about 80 nm under the same ball milling conditions. Also, a dispersion or variability about the typical sizes was closely similar for the four samples.

### Example 13

### Synthesis and Characterization of Nanoscale Adjuvants—Stability

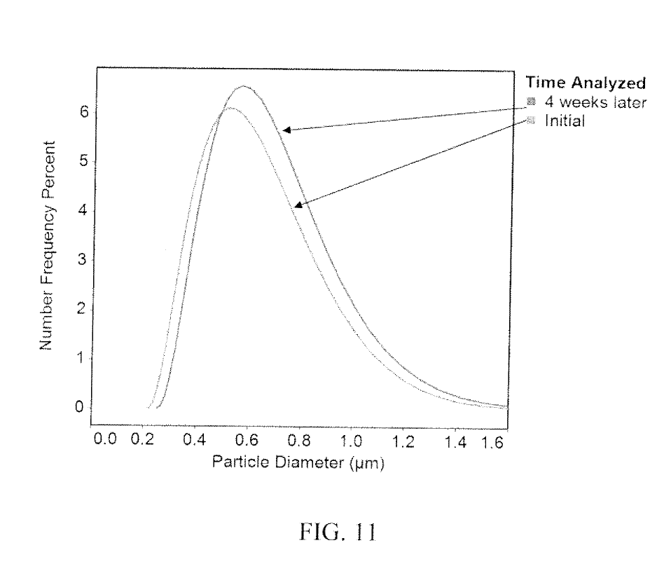

A nanoscale adjuvant was synthesized via ball milling and characterized in accordance with the methodology set forth in Example 1. To determine the stability in terms of cluster size and cluster size distribution, a sample including about 7 ml of a commercially available aluminum hydroxide hydrogel was subjected to ball milling. Size characteristics of the sample were measured substantially following ball milling. The sample was then stored in a plastic bottle following ball milling, and, after storage for about 4 weeks, its size characteristics were measured. Results of the measurements are illustrated in FIG. 11 as two cluster size distributions, with one distribution corresponding to the sample measured substantially following synthesis, and with another distribution corresponding to the same sample measured after storage for about 4 weeks.

As can be appreciated with reference to FIG. 11, high stability was observed in terms of cluster size and cluster size distribution with the passage of time. Referring to FIG. 11, size characteristics of the sample remained highly stable, with a relatively small increase of about 30 nm in typical cluster size after storage for about 4 weeks. Also, a dispersion or variability about the typical cluster size remained highly stable during the storage period. Without wishing to be bound by a particular theory, this observed stability in size characteristics indicates that little, or no, reaggregation occurred following ball milling.

### Example 14

### Characterization of Nanoscale Adjuvants—Effect of Cluster Size on Immunological Efficacy

To determine the effect of cluster size on immunological efficacy, an immunological study was carried out on differently sized adjuvants using Tetanus toxoid and toxin in mouse models. 76 male BALB/c mice (about 5 weeks old) were injected subcutaneously in their right and left inguinal areas (about 100 μL at each location) with vaccines. The study compared the efficacy of vaccines prepared using a commercially available alum (Alhydrogel, Cedarlane Laboratories Ltd., average cluster size on the order of 1 μm) and a ball milled alum (average cluster size=about 360 nm) as adjuvants. The study included three sets of control vaccinated groups, each including 4 mice. The control sets were distinguished by their vaccine type: commercially available alum alone, ball milled alum alone, and about 1.0 μg/mL of toxoid alone. All other vaccines were prepared by combining either commercially available alum or ball milled alum with toxoid concentrations of about 1.0 μg/mL, about 0.3 μg/mL, about 0.1 μg/mL, and about 0.03 μg/mL for injection in the remaining mice in groups of 8 mice each.

All mice were injected with their corresponding vaccine at Day 0. The vaccine injections were repeated at Day 28. Serum samples of each mouse were collected before Day 0, on Day 25, and on Day 45. All mice were finally challenged with a lethal dose of about 30 ng of Tetanus toxin (CalBiochem) in about 0.5 mL Hepes buffer on Day 42.

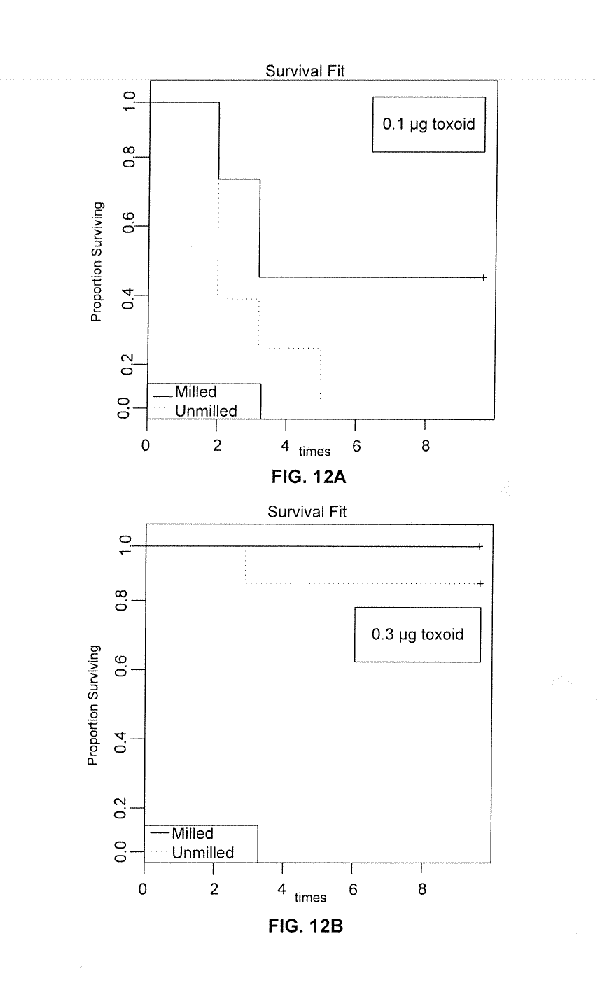

Results of the study were analyzed using the Kaplan-Meier method, with survival probabilities compared using the logrank test, as illustrated in FIG. 12A and FIG. 12B. For a vaccine dosing of about 0.1 μg/mL of toxoid, mice injected with vaccines including the commercially available alum and the ball milled alum had survival probabilities of about 0% and about 42%, respectively (p_{logrank test}=about 0.086). For a vaccine dosing of about 0.3 μg/mL of toxoid, mice injected with vaccines including the commercially available alum and the ball milled alum had survival probabilities of about 83% and 100%, respectively (p_{logrank test}=0.32). As such, vaccines prepared using the ball milled alum likely exhibit superior immunological efficacy relative to those prepared using the commercially available alum, indicating that the use of adjuvants with smaller cluster sizes can yield more efficacious vaccines.

### Example 15

### Characterization of Nanoscale Adjuvants—Effect of Cluster Size on Immunological Efficacy

To determine the effect of cluster size on immunological efficacy, an immunological study was carried out on differently sized adjuvants using ovalbumin (“OVA”) in mouse models. 76 male B6 mice were injected subcutaneously in their right and left inguinal areas (about 50 μL at each location) with vaccines. The study compared the efficacy of vaccines prepared using a commercially available alum (Novartis AG, average cluster size on the order of 1 μm) and a ball milled alum (average cluster size=about 100 nm, about 230 nm, about 330 nm, and about 880 nm) as adjuvants. The study included one control group of 5 mice that received no vaccine. All vaccines were prepared by combining either commercially available alum or ball milled alum with OVA concentrations of about 50 μg/mL, for injection in the remaining mice in groups of 5 mice each.

All mice were injected with their corresponding vaccine at Day 0. The vaccine injections were repeated at Day 7, 14, and 21. Serum samples of each mouse were collected on Day 6, 13, 20, and 27. All mice were finally harvested and tested by CD8-enzyme-linked immunospot technique (“ELISPOT”) on Day 28.

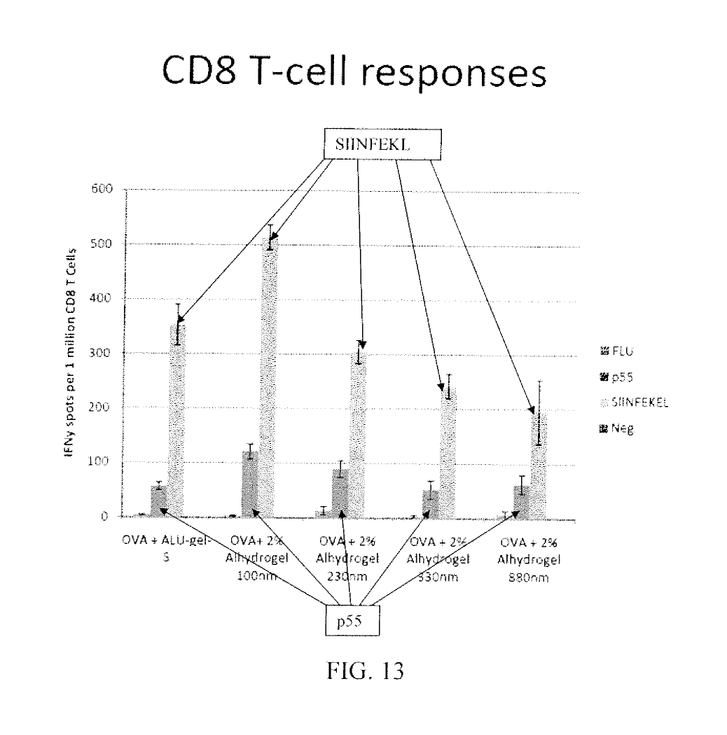

Results of CD8 T-cell response measurements are shown in FIG. 13. The CD8 T-cell response, as measured by the SIINFEKL and p55 OVA epitopes, increases with decreasing cluster size. As such, vaccines prepared using the ball milled alum likely exhibit superior immunological efficacy relative to those prepared using the commercially available alum, indicating that the use of adjuvants with smaller cluster sizes can yield more efficacious vaccines.

While the invention has been described with reference to the specific embodiments thereof, it should be understood by those skilled in the art that various changes may be made and equivalents may be substituted without departing front the true spirit and scope of the invention as defined by the appended claims. In addition, many modifications may be made to adapt a particular situation, material, composition of matter, method, or process to the objective, spirit and scope of the invention. All such modifications are intended to be within the scope of the claims appended hereto. In particular, while the methods disclosed herein have been described with reference to particular operations performed in a particular order, it will be understood that these operations may be combined, sub-divided, or re-ordered to form an equivalent method without departing from the teachings of the invention. Accordingly, unless specifically indicated herein, the order and grouping of the operations are not limitations of the invention.

What is claimed is:

1. A pharmaceutical composition, comprising: (a) a vaccine; and (b) a nanoscale adjuvant including an aluminum compound in the form of clusters having a peak size in the sub-micron range.

2. The pharmaceutical composition of claim 1, wherein the aluminum compound corresponds to one of aluminum hydroxide, aluminum phosphate, and alum.

3. The pharmaceutical composition of claim 1, wherein the peak size of the clusters is less than 800 nm.

4. The pharmaceutical composition of claim 1, wherein the peak size of the clusters is less than 600 nm.

5. The pharmaceutical composition of claim 1, wherein the peak size of the clusters is less than 400 nm.

6. The pharmaceutical composition of claim 1, wherein the peak size of the clusters is less than 200 nm.

7. The pharmaceutical composition of claim 1, wherein the clusters have a distribution of sizes with a standard deviation that is less than 70% relative to the peak size of the clusters.

8. The pharmaceutical composition of claim 7, wherein the standard deviation is less than 50% relative to the peak size of the clusters.

9. The pharmaceutical composition of claim 1, wherein at least one of the clusters includes constituent crystallites of the aluminum compound.

10. The pharmaceutical composition of claim 1, wherein the vaccine includes antigens that are coupled to the clusters.

11. The pharmaceutical composition of claim 10, wherein the antigens are adsorbed onto exposed surfaces of the clusters.

12. The pharmaceutical composition of claim 1, wherein the vaccine is derived from at least one of a bacterium, a virus, and a cancer cell.

13. The pharmaceutical composition of claim 1, wherein the vaccine corresponds to a cancer vaccine.

14. A method of stimulating an immune response in a patient, comprising: administering to the patient the pharmaceutical composition of claim 1.

15. The method of claim 14, wherein the patient is a human patient.

16. The method of claim 14, wherein a weight of aluminum per dose of the pharmaceutical composition is in the range of 1 μg/dose to 1.5 mg/dose.

17. A method of preparing a pharmaceutical composition, comprising:
providing an adjuvant including clusters having a peak size in the sub-micron range, wherein at least one of the clusters corresponds to an aggregate of grains of a compound; and
combining the adjuvant with a vaccine to form the pharmaceutical composition.

18. The method of claim 17, wherein the compound corresponds to an aluminum compound.

19. The method of claim 18, wherein providing the adjuvant includes ball milling the aluminum compound to form the clusters.

20. The method of claim 18, wherein the peak size of the clusters is in the range of 10 nm to 500 nm.
